(19) United States
(12) Patent Application Publication
(10) Pub. No.: US 2008/0071195 A1
(43) Pub. Date: Mar. 20, 2008

(54) Non-invasive tracking device and method

(76) Inventors: Alberto D. Cuellar, Houston, TX (US); Amir Sarvestani, Freiburg (DE); Jose Moctezuma de la Barrera, Freiburg (DE)

Correspondence Address: MCCRACKEN & FRANK LLP, 311 S. WACKER DRIVE, SUITE 2500, CHICAGO, IL, 60606, US

(21) Appl. No.: 11/522,644

(22) Filed: Sep. 18, 2006

(51) Int. Cl. A61B 19/00; A61B 5/11; G01C 25/00; G12B 13/00

(52) U.S. Cl. 600/595; 606/1; 128/897; 128/898; 73/865.4; 73/1.79

(57) ABSTRACT

A device for use with a surgical navigation system includes a body with an inner and outer wall that is shaped to envelope an extremity of a patient, a tracking device associated with the body in a fixed relation to the outer wall of the body, and a filler that fixably conforms to the space between the inner wall of the body and the extremity. The device can be used in orthopedic surgery.

### CROSS REFERENCE TO RELATED APPLICATIONS

Not applicable

### REFERENCE REGARDING FEDERALLY SPONSORED RESEARCH OR DEVELOPMENT

Not applicable

### SEQUENTIAL LISTING

Not applicable

### BACKGROUND OF THE INVENTION

1. Field of the Invention

This invention relates to a method to non-invasively establish a meaningful reference frame for a body region relative to which measurements are made. More particularly this invention exploits adjacency properties of the members of the extremities as well as the natural kinematics constraints of their joints to establish important parameters pertaining to the biomechanical axes of the members. Furthermore this invention handles means for temporarily affixing a tracking device at a distal end of an extremity member to enable accurate tracking of the underlying anatomical structures. This invention also describes alternate temporary constraining situations that can be used to establish a rigid transformation between a tracker and the anatomical structures.

2. Description of the Background of the Invention

Recently, the use of surgical navigation systems has become state-of-the-art for orthopedic and other types of surgery. One disadvantage of using a surgical navigation system is that tracking devices need to be affixed to a patients' extremity in order for the system to be able to sense the motion of that extremity. In a typical situation, the tracking device is affixed directly to the bone. There have been numerous proposals to minimize the invasion of the patient's anatomy. Some of the reasons that a minimally invasive system of attaching the tracking device to the patient is desirable is minimization of patient discomfort, minimization of potential for infection of other complication that results from disturbing the cortex of the bone.

These include the use of small posts for the tracking devices so that the disturbance of the cortex of the bone is minimized, systems to attach a tracking device to a portion of the bone that will be removed during the procedure, and the like. Each of these prior systems address the problem by making as small a disturbance as possible in the bone that will remain after the procedure. Because these systems are still somewhat invasive, they do not lend themselves to use in situations where the non-operative limb of a patient needs to be tracked for some purpose. For instance, it might be desirable to measure the range of motion of the non-operative leg during a hip replacement operation so that the surgeon can better understand of what changes need to be made to the operative leg so that the patient's quality of life is maximized.

In other situations it is desirable to inexpensively assess the biomechanics of the extremities before and after an operation is performed. Nowadays expensive methods as roentgen stereogrammetry allow for movement analysis of the limb. This method is used in research and is not suitable in a day to day situation for regular patient treatment.

### SUMMARY OF THE INVENTION

One aspect of the present invention relates to a medical device for use with a surgical navigation system that comprises a body shaped to envelope an extremity of a patient, where the body has an inner wall and an outer wall. The device also includes a tracking device associated with the body in a fixed relation to the outer wall of the body; and a filler that fixably conforms to the space between the inner wall of the body and the extremity.

A further aspect of the present invention relates to a method of attaching a tracking device to a patient in a non-invasive manner. The method comprises the steps of placing a body around an extremity of the patient; non-invasively securing the body to the extremity so that the body is fixed relative to the extremity; and associating a tracking device to the body so that the tracking device is fixed relative to the body.

A still further aspect of the present invention concerns a method for establishing a coordinate system of an anatomical structure with reference to neighboring structures and for establishing spatial relationships between the anatomical structure and the neighboring structures or surgical devices in a non invasive manner. The method comprises the steps of placing an enveloping body around a portion of an extremity of a patient, and associating a tracking device with the enveloping body. The method further comprises the steps of manipulating the extremity using natural extremity joint constraints to create rigid transformation situations to access parameters of adjacent limb members of the extremity; and tracking the tracking device during the manipulation to establish the coordinate system.

Other aspects and advantages of the present invention will become apparent upon consideration of the following detailed description.

### BRIEF DESCRIPTION OF THE DRAWINGS

FIG. 1 is a representation of one embodiment of the present invention;

FIG. 2 is a schematic cross-section generally taken along the line 2-2 in FIG. 1;

FIG. 3 is a representation of a further embodiment of the present invention;

FIG. 4 is a schematic cross-section generally taken along the line 4-4 in FIG. 3;

FIG. 5 is a representation of a still further embodiment of the present invention;

FIGS. 6A to 6C are a schematic representation of the use of present invention in knee surgery; and

FIG. 7 is a representation an additional embodiment of the present invention.

### DETAILED DESCRIPTION OF THE PREFERRED EMBODIMENTS

The following detailed description primarily uses the foot as an example of a distal end of an extremity. The present invention can also be used with other extremities.

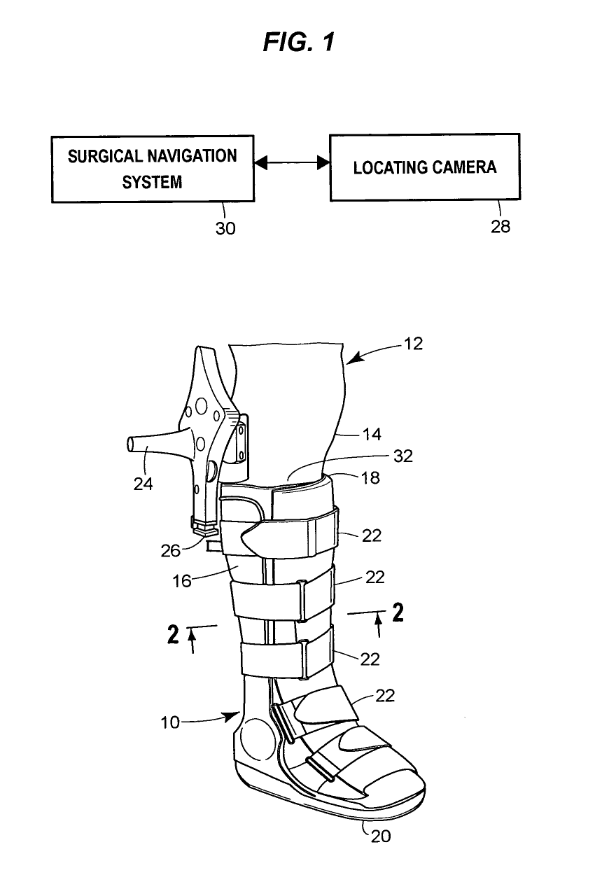

FIG. 1 shows an embodiment of the present invention that includes a boot 10 worn by patient 12. The boot 10 extends sufficiently far up the leg 14 of the patient 12 so that the boot 10 will move with the leg 14, and more importantly with the tibia within the leg 14 so that there is negligible movement of the boot 10 relative to the leg 14, and in particular the tibia within the leg 14. In this regard, the boot 10 has a body 16 that extends from a proximal end 18 to a distal end 20. The distal end 20 of the body 16 is shaped to immobilize the foot to minimize movement relative to the tibia. In a typical situation, the body 16 will be formed from a rigid material such as hard plastic, stiff leather or structures that include metal reinforcements to immobilize the foot relative to the tibia. In this regard, the boot 10 can be any of the known immobilizing devices for the foot and lower leg provided that the proximal end 18 of the boot 10 extends part way up the leg 14.

The boot 10 also can have one or more straps 22 that can be used to firmly affix the boot 10 to the leg 14. Other types of closures for the boot 10 can be used in place of straps 22 so long as the boot 10 can be firmly placed on the foot and lower lag 14 of the patient 12. These closures include clamps, buckles, adhesive tape, and the like. A tracking device 24 is attached or associated to the body 16 using a coupling 26. The particular tracking device can be any of the known types of tracking devices, including active optical devices that have emitters such as LEDS that send a signal to a locating device, passive optical devices that reflect light back to a locating device, magnetic devices, acoustic devices or inertial sensor devices. Any known tracking technology can be used as tracking device 24. The tracking device should be attached to the body 16 in such a way that there is no relative movement between the body 16 and the tracking device 24. The particular nature and structure of the coupling 26 is not important so long as there is no relative movement between the body 16 and the tracking device 24. If the tracking device 24 is an optical tracking device, the tracking device 24 will communicate with a locating camera 28 that communicates with a diagnostic or therapeutic system 30. If the tracking devise 24 is an inertial tracking device, the tracking device 24 will directly communicate with the diagnostic or therapeutic system 30.

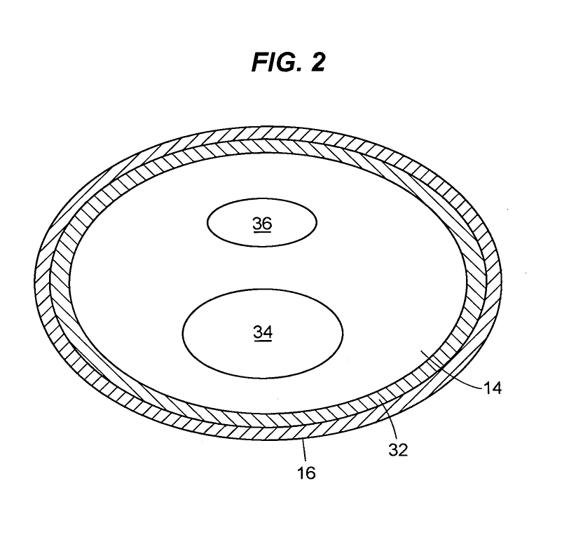

As shown in FIG. 2, the boot 10 also has an inner layer 32 that snuggly conforms to the surface of the leg 14 to hold the leg 14 in place relative to the body 16. The inner layer 28 can be any material that can conform to the shape of leg 14. Suitable materials include foams, gels, compressible solids, and the like. Inner layer 32 could also be a foam layer that is formed in situ by injecting foaming material into the boot 10 after the leg 14 has been placed within the boot 10. In this case, the design of the boot 10 should allow the body 16 of the boot 10 to be removed without requiring that the inner layer 32 to be removed at the same time so that the inner layer 32 can be separately removed from the leg 14 after the body 16 has been removed. For instance the boot 10 could be made up of a body 16 that has two halves that are joined together in the front by straps 22 or other similar connecting devices. The tibia 34 and fibula 36 are held firmly in place relative to the body 16 by the inner layer 28 acting on the surface of the leg 14.

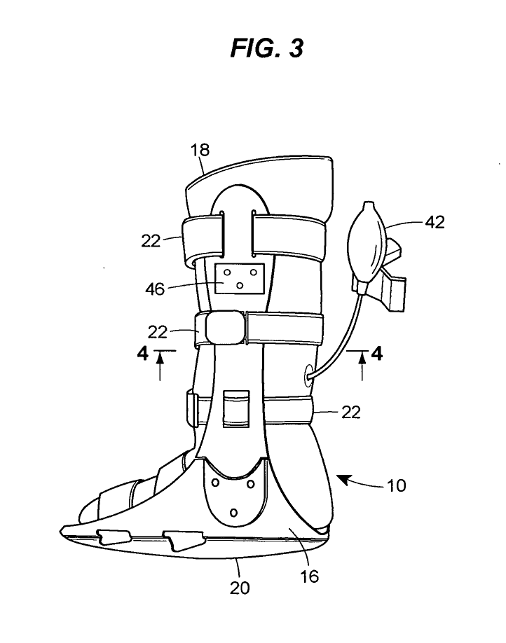

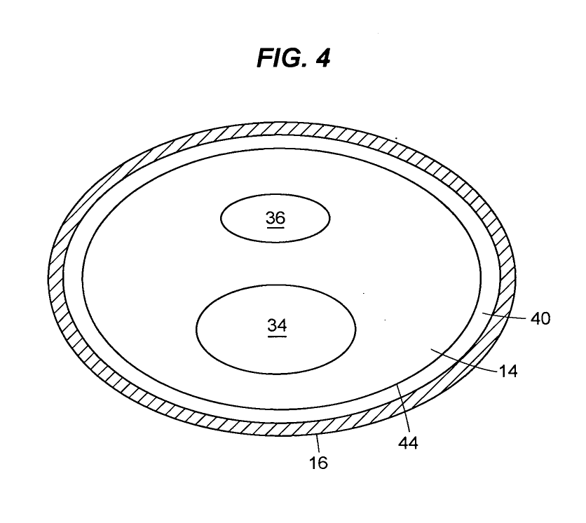

FIGS. 3 and 4 show an additional embodiment of the preset invention where the boot 10 has an inner layer 32 that is a bladder 40. The bladder 40 can be filled with any fluid material including air using a pump 42. In this embodiment, the pump 42 will fill the bladder 40 so that there is no relative movement between the leg 14 and the body 16 of the boot 10. In order to minimize any relative movement, an optional inner wrapping 44 can also be used with this embodiment or the other embodiments of the present invention. The wrapping 44 can be any suitable material that is acceptable for skin contact and that will provide additional friction between the leg 14 and the inner layer 32. Suitable commercially available materials include Coban sold by 3M. A tracking device 46 is integral with the body 16 of the boot 10.

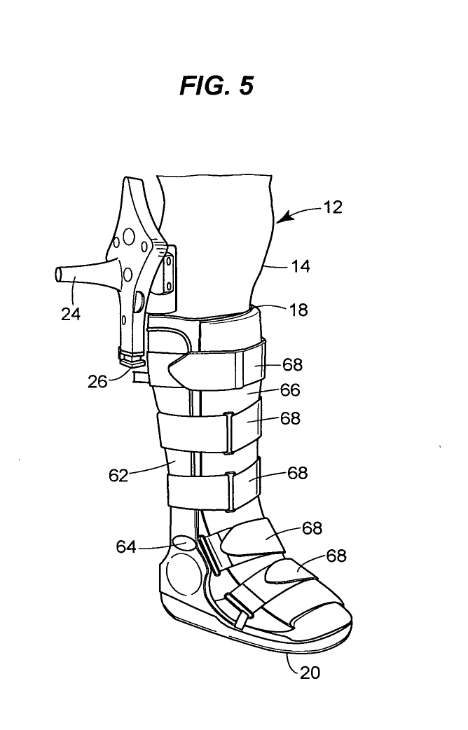

FIG. 5 is a still further embodiment of the present invention. In this embodiment, a boot 60 has a body 62 similar to the construction of the body 16 above. However, in this embodiment, the body 62 has openings 64 (only one of which is shown) that extend through the body 62 and the inner layer (not shown) that is substantially similar to the inner layer 32 described above. The location of the openings 64 are such that the medial and lateral malleolus can be located. For certain types of procedures, the location of the medial and lateral malleolus is used to locate other anatomical axes and landmarks. The location of the medial and lateral malleolus is done is a conventional and known manner. In addition, boot 60 has a front panel 66 that is held in place by removable straps 68. Front panel 66 is removable so that the leg 14 can be easily inserted into the boot 60 and firmly held in place by the interaction of body 62 and front panel 66 as well as any inner layer contained within the boot 60.

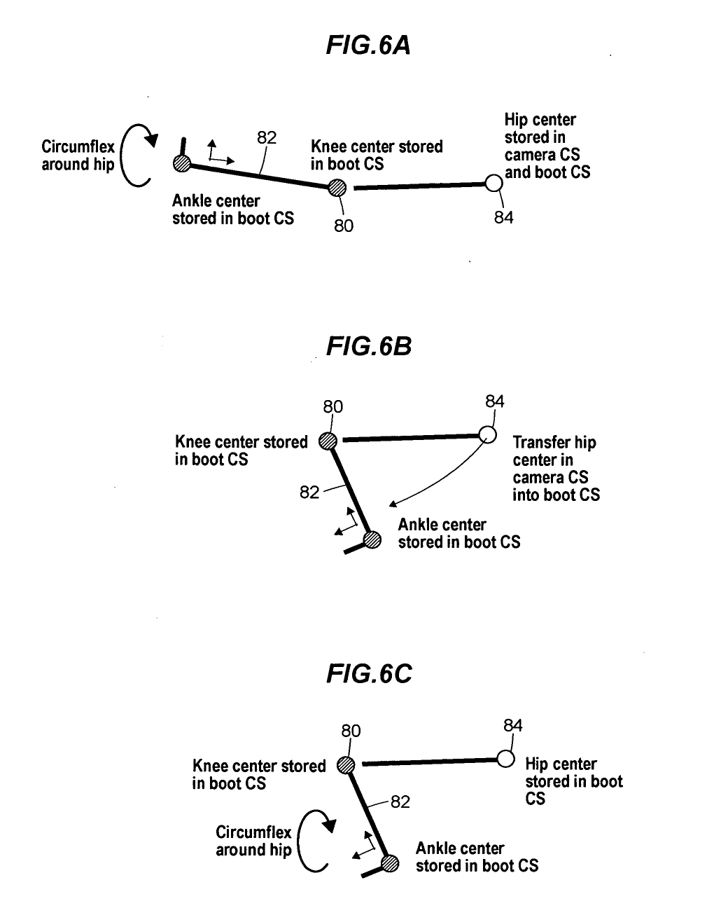

With reference to FIGS. 6A to 6C, the use of the boot 60 will now be described in the context of a knee replacement surgical procedure. The particular approach to the procedure for the complete or partial knee replacement is not important to the use of the present invention, and the present invention can be used with any diagnostic or therapeutic approach to joint arthroplasty or trauma surgery of the extremities. Natural anatomical constraints of the joints in conjunction with kinematics analysis algorithms are used to establish local meaningful anatomical reference frames to quantitatively assess a biomechanical condition or aid the surgeon during the execution of a therapeutic measure to achieve a desired outcome. In the case of the knee arthroplasty the relationship of the biomechanical axes of the tibia and femur need to be established in all planes in order to guide the necessary resection and ligament releases and ultimately position the prosthetic components.

The first parameter to be acquired is the hip center 80 as one of the points with which the mechanical axis of the femur 82 is constructed. For this the knee 84 is held in full extension to make sure that there is a constant transformation between the tibia 86 and the femur 82 of the patient 12. Here the ligaments and other structures prevent the limb from hyperextending. This creates a reproducible constraint of all six degrees of freedom between tibia 86 and femur 82. This relationship enables a surgeon to derive structures pertaining to the femur 82 relative to the tibia 86. The hip center 80 is calculated by circumflexing the hip with the knee 84 in full extension. Typically the surgeon will support the calf 90 with their hand while this is being done. The hip center 80 is stored by the diagnostic or therapeutic system 30 in both the boot coordinate system and the camera coordinate system.

Depending on the training and the preferences of the surgical team, there are different methods to determine the knee center. Some of these methods can be performed in a non-invasive manner. Other methods involve opening the knee capsule to directly digitize points. One invasive method is the direct digitization of the knee center using a tracked pointer device that traces the surfaces of the knee structure after the knee capsule has been opened. An alternate or supplemental method, locates the epicondyles of the femur 82 using a pointer. This can be done either invasively with the knee opened or using the surface of the skin with the knee closed. The knee center is considered as the mid-point of the epicondyles.

A third method uses motion recording of the knee flexion in order to calculate the flexion axis plus digitizing the tibial tuberosita. The digitized tibial tuberosita are projected onto the flexion axis to define the knee center. This method can be done with the knee 84 opened or with the knee 84 closed. A fourth method also uses motion recoding of the knee flexion to calculate the flexion axis plus digitizing the epicondyles. The mid-point of the epicondyles is projected onto the flexion axis to define the knee center. This fourth method can be done with the knee 84 open or with the knee 84 closed.

Next, the ankle center is located using the medial and lateral malleolus in a typical manner well know to those skilled in the art. An alternative method would be to use the well known transformation between the boot tracker reference frame and the ankle center position of the boot. This method does not require the step of maleoli digitization but does require a constant relationship between the boot tracker and the ankle center location within the boot.

If a non-invasive technique has been used as noted above, it is only at this point that any invasion of the body occurs. Here, the surgeon will make an incision at the knee to open the knee compartment and begin the replacement procedure. If either the knee center or the AP axis or the epicondyles have not been determined non-invasively as described above, direct digitization of corresponding knee landmarks in a known manner can be used to determine them.

At this point, all of the relevant alignment parameters and relationship between the tibia and femur are either known or can be determined by the diagnostic or therapeutic system 30. These parameters are typically varus/valgus alignment, internal/external rotation and flexion range. Optionally, varus/valgus laxity in extension can be determined if desired. Additional recordings are needed to determine this parameter. One method involves digitizing the boot tracker when applying varus stress to the knee and digitizing the boot tracker when applying valgus stress to the knee. The knee must be in full extension during this determination. A further optional parameter would be range of motion of the knee joint. This parameter is either directly determined by flexing and extending the knee to the maximum while keeping the hip stable and recoding the movement, or by recording the hip center with motion analysis first with the knee in full extension and then with the knee in full flexion.

The balance of the replacement procedure will proceed in a known manner using the approach with which the surgeon is most familiar. In one method, the surgical instruments, such as guides, jigs and cutting blocks, can be tracked and navigated in a non-invasive or minimally invasive manner using methods disclosed in co-pending application Ser. No. 11/251,044. Filed Oct. 14, 2005, entitled, system and method for bone resection, the disclosure of which is hereby incorporated by reference. After implantation of the prosthetic components the same methods as disclosed above can be used to obtain the newly installed biomechanical configuration of the limb. The diagnostic or therapeutic system 30 will be able to calculate the final varus/valgus alignment, internal/external rotation as well as flexion range of motion of the pre-operative or the post-operative situation.

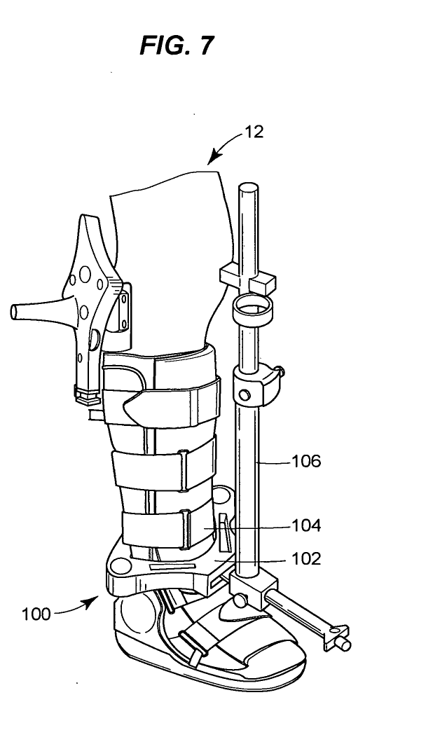

In another embodiment as shown in FIG. 7, the boot 100 can include a clamp 102 at the anterior distal section 104 to allow an extra-medullary tibia rod 106 to be attached to the boot 100. The use of the extra- medullary tibia rod 106 is well known and will not be further discussed here.

One advantage in using the boot of the present invention, is that the opposite side (non-operative) leg can also be evaluated in a noninvasive manner for comparison purposes during the replacement procedure. The same non-invasive procedure as described above is repeated for the non-operative leg. This is also outside joint arthroplasty of immense value as e.g. in trauma and reconstructive surgery where natural biomechanics of the contralateral side are often otherwise impossible to reproduce intra-operatively without significant technical and logistical efforts such as intra-operative CT scans.

In a similar manner, the necessary anatomical locations can be determined for hip replacement surgery. In this case the main parameter of interest is leg length. After attaching the boot, the hip center is determined by circumflexing the hip with the knee in full extension. The ankle center is then digitized as described above relative to determination of the knee center. The distance between ankle center and hip center 80 serves as the initial leg length. The procedure is repeated after the hip implant procedure has been complete to calculate the final leg length as the distance between the hip center 80 and the ankle center in the final alignment. Alternatively to the ankle center, any other anatomical point on the femur or tibia can be used as the distal reference. Optionally, the procedure can also be applied to the opposite leg to use the leg length of the opposite leg as a reference for the leg length of the treated leg.

A further application at the lower extremities is the treatment of femoral or tibia fractures. In these procedures a major difficulty is restoration of the correct leg length after fracture reduction and implant fixation. A boot is attached to the non-fractured leg. By motion analysis with the knee in full extension the hip center 80 is determined and stored in the coordinate system of the boot tracker. The distance between the hip center 80 and the boot is used as the reference for leg length. Alternatively, the ankle center can be determined as described above to calculate leg length as the distance between the hip center and the ankle center. At this point in the procedure, the boot is attached to the fractured leg. After nail insertion and preliminary reduction, a motion analysis with the knee in full extension is performed in the same manner as for the non-fractured leg to calculate the leg length for the leg with the fracture. The leg length is compared to the length of the opposite non-fractured leg. The surgeon can then make adjustments to the reduction to obtain matching leg lengths.

Also, using a suitable shell that is shaped to be placed around the forearm, the same technique as described above can be used for shoulder surgery as well. In general, the stabilization method of the tracker to the anatomy is an encapsulating conforming shell with or without active or passive layers that conform preferably around the metaphysical aspects of the members of a limb. These anatomical regions usually possess enough local geometrical irregularities that allow the shell device to lock onto the underlying anatomical structures so that there is no translational or rotational movement between the shell and the underlying anatomical structures. Even if adipose tissue attenuates the geometric metaphysical irregularities so that the shell can not be properly locked onto the underlying anatomical structure, it is possible to create a pseudo constraint by flexing the joint onto which the shell will be attached. The flexed joint now provides enough conforming areas to provide a stable and rigid configuration.

### INDUSTRIAL APPLICABILITY

This invention is usable to assist in joint replacement surgeries without adding to the necessary invasion of the body.

Numerous modifications to the present invention will be apparent to those skilled in the art in view of the foregoing description. Accordingly, this description is to be construed as illustrative only and is presented for the purpose of enabling those skilled in the art to make and use the invention and to teach the best mode of carrying out same. The exclusive rights to all modifications which come within the scope of the appended claims are reserved.

I/We claim:

1. A medical device for use with a surgical navigation system comprising:
a body shaped to envelope an extremity of a patient, the body having an inner wall and an outer wall;
a tracking device associated with the body in a fixed relation to the outer wall of the body; and
a filler that fixably conforms to the space between the inner wall of the body and the extremity.

2. The medical device of claim 1 wherein the body is rigid.

3. The medical device of claim 1 wherein the filler is an inner liner of the body that conforms to the extremity.

4. The medical device of claim 1 that includes an opening in the body to enable the determination of an anatomical landmark.

5. The medical device of claim 1 wherein the extremity is the leg and the body is in the shape of a boot.

6. The medical device of claim 1 wherein the filler is a bladder that can be filled with a fluid.

7. The medical device of claim 1 wherein the tracking device is an inertial sensor.

8. The medical device of claim 1 wherein the tracking device is an optical sensor tracked by an optical tracking system.

9. The medical device of claim 1 wherein the tracking device is an electromagnetic sensor tracked by an electromagnetic tracking system.

10. The medical device of claim 1 wherein the tracking device is integral to the outer wall.

11. The medical device of claim 1 wherein the tracking device is mounted on the outer wall.

12. The medical device of claim 11 wherein the tracking device is mounted using a rigid connector attached to the outer wall.

13. The medical device of claim 1 that includes a clamp for an extra-medullary tibia rod.

14. A method of attaching a tracking device to a patient in a non-invasive manner, the method comprising the steps of:
placing a body around an extremity of the patient;
non-invasively securing the body to the extremity so that the body is fixed relative to the extremity; and
associating a tracking device to the body so that the tracking device is fixed relative to the body.

15. The method of claim 14 wherein the non-invasive securing is done by using an inner layer that conforms to the extremity.

16. The method of claim 14 wherein the non-invasive securing is done by using a bladder that can be filled with fluid.

17. The method of claim 14 wherein the tracking device is used during a knee replacement procedure to determined the necessary anatomical landmarks.

18. The method of claim 14 wherein the tracking device is used during a hip replacement procedure to determined the necessary anatomical landmarks.

19. The method of claim 14 wherein the associating of the tracking device is performed by attaching the tracking device to a connector that is affixed to the body.

20. A method for establishing a coordinate system of an anatomical structure with reference to neighboring structures and for establishing spatial relationships between the anatomical structure and the neighboring structures or surgical devices in a non invasive manner, the method comprising the steps of:
placing an enveloping body around a portion of an extremity of a patient,
associating a tracking device with the enveloping body,
manipulating the extremity using natural extremity joint constraints to create rigid transformation situations to access parameters of adjacent limb members of the extremity; and
tracking the tracking device during the manipulation to establish the coordinate system.

21. The method of claim 20 wherein the extremity is a leg and the portion of the anatomy is an ankle.

22. The method of claim 21 wherein the manipulation is done with the leg in extension.

23. The method of claim 21 wherein the manipulation is done with the leg in flexion.
